(19) United States
(12) Patent Application Publication
(10) Pub. No.: US 2004/0258631 A1
(43) Pub. Date: Dec. 23, 2004

(54) Oral care compositions exhibiting antiplaque and breath freshening properties

(76) Inventors: Thomas J. Boyd, Somerset, NJ (US); Guofeng Xu, Princeton, NJ (US); Abdul Gaffar, Princeton, NJ (US); David B. Viscio, Monmouth Jct., NJ (US)

Correspondence Address: Colgate-Palmolive Company, 909 River Road, P.O. Box 1343, Piscataway, NJ, 08855-1343, US

(21) Appl. No.: 10/601,474

(22) Filed: Jun. 23, 2003

(51) Int. Cl. A61K 7/16; A61K 7/26

(52) U.S. Cl. 424/49

(57) ABSTRACT

A stable aqueous antiplaque oral composition containing (a) an antibacterial ester compound having the formula

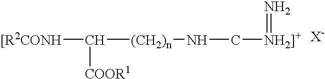

wherein R¹ is an alkyl chain of 1 to 8 carbon atoms, and R² is an alkyl chain of 6 to 30 carbon atoms and X is an anion, and, a silica compound precoated with an ethoxylated hydrogenated castor oil.

### BACKGROUND OF THE INVENTION

1. Field of the Invention

The present invention relates to an oral care composition which contains a antibacterial ester compound which composition is effective in retarding bacterial plaque accumulation on teeth and more particularly to a dentifrice composition containing a antibacterial ester compound which achieves plaque reduction with superior breath freshening characteristics.

2. The Prior Art

Halitosis, the technical term for bad breath, or Fetor ex Oris, is an undesirable condition. As a matter of fact, everyone, excluding the very young, occasionally has bad breath, with approximately 25% suffering on a regular basis and the problem tends to get worse and more frequent as one gets older. The problem seems to be evenly split between men and women. Bad breath results when proteins from the food we eat and saliva debris are broken down by bacteria. Even the cleanest mouth hosts millions of bacteria which have the potential to decompose these protein-containing particles left in the mouth. This bacterial population forms foul smelling products, called volatile sulfur compounds (VSC)—such as hydrogen sulfide (“rotten eggs”) and methyl mercaptans (“skunk smell”) and other odorous and bad tasting compounds. Up to 80-90% of bad breath that originates in the mouth is by this mechanism.

Dental plaque or plaque bio-film is a soft deposit that forms on teeth and is comprised of an accumulation of bacteria and salivary as well as food by-products. Plaque adheres tenaciously at the points of irregularity or discontinuity, e.g., on rough calculus surfaces, at the gum line, on tongue surface and within crevices, and the like. Besides being unsightly, plaque is implicated in the occurrence of gingivitis and other forms of periodontal disease.

A wide variety of antibacterial agents have been suggested in the art to retard plaque formation and the oral infections and dental disease associated with plaque formation. For example U.S. Pat. No. 5,874,068 and UK 1352420 discloses that arginine derivative compounds exhibit antibacterial activity when used in oral compositions such as mouthrinses to counter plaque formation by bacterial accumulation in the oral cavity.

Arginine derivative compounds and their salts in particular show excellent inhibitory effect against microorganisms which possess relatively strong resistance to bacterial such as S. aureus, S. mutans, F.nucleatum which are involved in plaque formation on teeth.

Although the arginine derivative compounds disclosed in the prior art are effective antibacterial agents, when these compounds are included in silica containing dentifrice it was discovered that when the dentifrice was applied to the teeth, the bioavailability of the arginine derivative compound was reduced to a level whereby little antiplaque benefit was achieved. Investigation of this problem led to the discovery that compounds such as abrasives and thickeners such as silica compounds conventionally used in the preparation of dentifrice compositions were the factor responsible for the impairment of the antiplaque efficacy of the arginine derivative compound.

Thus, there is a clear need in the art to formulate a dental product capable of delivering an arginine derivative compound antiplaque agent whereby the ingredients used to prepare the dentifrice composition do no inhibit the bioavailability of the antiplaque agent so that optimum antiplaque benefits result.

### SUMMARY OF THE INVENTION

The present invention encompasses a dentifrice composition containing in an orally acceptable vehicle a combination of (1) an arginine derivative antibacterial compound and an abrasive compound whereby superior reduction of plaque accumulation is accompanied by enhanced malodor reduction, the arginine derivative compound having the formula

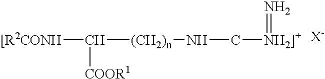

where R¹ is an alkyl chain of 1 to 8 carbon atoms, and R² is an alkyl chain of 6 to 30 carbon atoms and X is an anion, and (2) a silica compound, the silica compound having been first coated with an ethoxylated hydrogenated castor oil, whereby superior reduction of plaque accumulation is accompanied by enhanced malodor reduction.

In the practice of the present invention the dentifrice composition containing the arginine antibacterial compound and an ethoxylated hydroxylated castor oil coated silica abrasives and thickeners is formulated as a paste or gel using a vehicle containing a safe and effective amount of the antibacterial arginine derivative compound wherein the presence of the silica compound does not inhibit the bioavailability of the antibacterial arginine derivative compound.

Dentifrice Vehicle

The orally-acceptable dentifrice vehicle used to prepare the dentifrice composition comprises a water-phase, containing a humectant therein. The humectant is preferably glycerin, sorbitol, xylitol, and/or propylene glycol of molecular weight in the range of 200 to 1,000. Other humectants, such as polyethylene glycol, and mixtures thereof may also be employed. The humectant concentration typically totals about 5 to about 70% by weight of the oral composition.

Reference hereto to sorbitol refers to the material typically commercially available as a 70% aqueous solution. Water is present typically in amount of at least about 10% by weight, and generally about 15 to 30% by weight of the oral composition. Water employed in the preparation of commercially suitable toothpastes should preferably be deionized and free of organic impurities. These amounts of water include the free water which is added plus that which is introduced with other materials such as with sorbitol.

Antibacterial Ester

In the above identified antibacterial ester formula, R²CO may be a natural system mixed fatty acid residue such as coconut oil fatty acid, tallow fatty acid residue and the like, or a mono-fatty acid residue such as lauroyl, myristyl, stearoyl and the like, the lauroyl group being preferred.

Examples of antibacterial ester salts of the above identified formula include inorganic acid salts such as hydrochloride, sulfate or an organic salt such as acetate, tautarate or citrate, the chloride salt being preferred.

Examples of antibacterial ester compounds preferred in the practice of the present invention are antibacterial ester compound of the above-identified formula wherein n in the formula equals 3 useful in the practice of the present invention include N⁶⁰ -cocoyl-L-arginine methyl ester, N^{α}-cocoyl-L-arginine ethyl ester, N^{α}-cocoyl-L-arginine propyl ester, N^{α} stearoyl-L-arginine methyl ester, N^{α} stearoyl-L-arginine ethyl ester hydrochloride. The term “cocoyl” is an abbreviation for coconut oil fatty acid residue, and chloride salts of these compounds, these ester compounds and the salts thereof being referred to in this specification as arginine derivative compounds. The salt of the arginine derivative compound, ethyl lauroyl arginine, is preferred for use in the practice of the present invention.

The antibacterial ester of the present invention is present in the aqueous oral compositions of at a concentration of about 0.05 to about 2.0% by weight and preferably about 0.075 to about 1% by weight.

Silica Compounds

Silica abrasives useful in the practice of the present invention include silica gels and precipitated amorphous silicas. These silicas are colloidal particles having an average particle size ranging from about 3 microns to about 12 microns, and more preferably between about 5 to about 10 microns and a pH range from 4 to 10 preferably 6 to 9 when measured as a 5% by weight slurry.

Illustrative of silica abrasives useful in the practice of the present invention are marketed under the trade designation Sylodent XWA by Davison Chemical Division of W.R. Grace & Co., Baltimore, Md. 21203. Sylodent 650 XWA, a silica hydrogel composed of particles of colloidal silica having a water content of 29% by weight averaging from about 7 to about 10 microns in diameter.

Other abrasives used in the practice of the present invention include precipitated silicas having a mean particle size of up to about 20 microns, such as Zeodent 115, marketed by J.M. Huber Chemicals Division, Havre de Grace, Md. 21078, or Sylodent 783 marketed by Davison Chemical Division of W.R. Grace & Company.

The silica abrasive materials may be used individually as the sole abrasive in preparing the dental composition of the present invention or in combination with other known dentifrice abrasives such as sodium metaphosphate, dihydrated dicalcium phosphate, calcined alumina. The total quantity of abrasive present in the dentifrice compositions of the present invention is at a level of from about 5% to about 60% by weight, preferably from about 10% to about 55% by weight when the dentifrice composition is a toothpaste.

Silica compounds which function as thickening agents which may be used in the practice of the present invention include colloidal silica compounds available under the trade designation Cab-o-sil manufactured by Cabot Corporation and distributed by Lenape Chemical, Bound Brook, N.J.; Zeodent 165 from J.M. Huber Chemicals Division, Havre de Grace, Md. 21078; and Sylodent 15, available from Davison Chemical Division of W.R. Grace Corporation, Baltimore, Md. 21203.

Ethoxylated Hydrogenated Castor Oil

The ethoxylated hydrogenated castor oils used to precoat the silica compounds prior to their incorporation into the dentifrice of the present invention are prepared by hydrogenating castor oil and treating the hydrogenated product with from about 10 to about 200 moles of ethylene glycol. These ethoxylated hydrogenated castor oils are known by the non-proprietary name of PEG hydrogenated castor oils, in accordance with dictionary of the Cosmetics, Toiletries and Fragrance Association, 3rd Edition which name is used in conjunction with a numeric suffix to designate the degree of ethoxylation of the hydrogenated castor oil product, i.e., the number of moles of ethylene oxide added to the hydrogenated castor oil product. Suitable PEG hydrogenated castor oils include, PEG 16, 20, 25, 30, 40, 50, 60, 80, 100, and 200. In a preferred embodiment, the PEG 40 hydrogenated castor oil surfactant is Cremophor RH40, a commercially available product from BASF-Wyandotte, Parsippany, N.J. Ethoxylated hydrogenated castor oil is coated on the silica compounds used in the preparation of the compositions of the present invention at a castor oil to silica weight ratio of about 1:10 to 1:2.

Surfactant

Surfactants useful in the practice of the present invention include nonionic and zwitterionic surfactants. Suitable nonionic surfactants useful in the present invention include condensates of sorbitan esters of fatty acids with ethylene oxide (polysorbates) such as sorbitan mono-oleate with from about 20 to about 60 moles of ethylene oxide (e.g., “Tweens”, a trademark of ICI US, Inc.). Particularly preferred Polysorbates are Polysorbate 20 (polyoxyethylene 20 sorbitan monolaurate, Tween 20) and Polysorbate 80 (polyoxyethylene 20 sorbitan mono-oleate, Tween 80). Other nontonic surfactants include poly(oxyethylene)-poly(oxypropylene) block copolymers. Such copolymers are known commercially by the non-proprietary name of poloxamers, which name is used in conjunction with a numeric suffix to designate the individual identification of each copolymer. Poloxamers may have varying contents of ethylene oxide and propylene oxide which results in poloxamers which have a wide range of chemical structures and molecular weights. A preferred poloxamer is Poloxamer 407, sold under the tradename Pluronic F-127 by BASF, Wyandotte, Parsippany, N.J.

Zwitterion surfactants useful in the practice of the present invention particularly betaine surfactants, include surfactants disclosed in U.S. Pat. No. 5,180,577 incorporated herein by reference. Typical alkyl dimethyl betaines include decyl betaine 2-(N-decyl-N,N-dimethylammonio) acetate, cocobetaine or 2-(N-coc-N,N-dimethyl ammonio) acetate, myristyl betaine, palmityl betaine, lauryl, betaine, cetyl betaine, cetyl betaine, stearyl betaine, etc. The amido betaines are exemplified by cocoamidoethyl betaine, cocoamidopropyl betaine, laurmidopropyl betaine and the like. The preferred betaine is the cocoamidopropyl betaine.

The surfactant(s) is present in the oral composition of the present invention in the range from about 0.1% to about 5% by weight preferably from about 0.6% to about 2.0% by weight.

Thickening Agents

Thickeners used in the compositions of the present invention other than silica thickeners include natural and synthetic gums and colloids. Suitable thickeners include naturally occurring polymers such as carrageenans, xanthan gum, synthetic thickener such as polyglycols of varying molecular weights sold under the tradename Polyox and cellulose polymers such as hydroxyethyl cellulose and hydroxpropyl cellulose. Other inorganic thickeners include natural and synthetic clays such as hectorite clays, lithium magnesium silicate (laponite) and magnesium aluminum silicate (Veegum).

The thickening agent is present in the dentifrice composition in amounts of about 0.1 to about 10% by weight, preferably about 0.5 to about 4.0% by weight.

Fluoride

The dentifrice composition of the present invention may also contain a source of fluoride ions or fluorine-providing component, as anticaries agent in amount sufficient to supply about 25 ppm to 5,000 ppm of fluoride ions and include inorganic fluoride salts, such as soluble alkali metal salts. For example, preferred fluoride sources which are compatible with enzymes present in the composition are sodium fluoride, potassium fluoride, sodium fluorosilicate, ammonium fluorosilicate, as well as tin fluorides, such as stannous fluoride and stannous chloride. Sodium fluoride is preferred.

Antitartar Agents

In addition to fluoride compounds, there may also be included antitartar agents such as zinc salts including zinc chloride, zinc citrate and zinc gluconate which are compatible with the antibacterial ester. These antitartar agents are included in the dentifrice composition at a concentration of about 1 to about 5% by weight.

Other agents compatible with antibacterial esters also be included in the oral composition of the present invention such as antitartar agents as for example cationic polyphonates such as water soluble quaternary aminoalkylene phosphonic compounds as disclosed in U.S. Pat. No. 4,118,472, the disclosure of which is herein incorporated by reference. These antitartar agents may be included in the oral composition of the present invention at a concentration of about 0.1 to about 5% by weight.

Antitartar agents which are not compatible with antibacterial esters such as pyrophosphate and polyphosphate salts may be included in one component of a dual component oral composition system in which a first component contains the antibacterial ester and the second component contains the incompatible antitartar salt, the first and second components being maintained separate from each other until dispersed and combined for application to the teeth.

Flavor

The dentifrice composition of the present invention may also contain a flavoring agent. Flavoring agents which are used in the practice of the present invention include essential oils as well as various flavoring aldehydes, esters, alcohols, and similar materials. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Of these, the most commonly employed are the oils of peppermint and spearmint.

The flavoring agent is incorporated in the dentifrice composition at a concentration of about 0.1 to about 5% by weight and preferably about 0.5 to about 1.5% by weight.

Other Ingredients

Various other materials may be incorporated in the dentifrice compositions of this invention, including desensitizers, such as potassium nitrate; whitening agents; preservatives; silicones; and chlorophyll compounds. These additives, when present, are incorporated in the dentifrice composition in amounts which do not substantially adversely affect the properties and characteristics desired.

Preparation of the Dentifrice

The preparation of dentifrices is well known in the art. More specifically, to prepare a dentifrice of the present invention, generally the humectants e.g. glycerin, sorbitol, propylene glycol, are dispersed in water in a conventional mixer under agitation. Into the dispersion are added the arginine derivative compound, organic thickeners, such as carageenan, any salts, such as sodium fluoride anticaries agents; and any sweeteners. The resultant mixture is agitated until a homogeneous gel phase is formed. Into the gel phase are added a pigment such as TiO₂, and any acid or base required to adjust the pH to 6 to 7. These ingredients are mixed until a homogenous phase is obtained. The mixture is then transferred to a high speed/vacuum mixer; wherein, the surfactant ingredients are added to the mixture as well as the silica compounds such as silica abrasive Zeodent 115 and silica thickener Zeodent 165 both compounds being precoated with an ethoxylated hydrogenated castor oil. The mixture is then mixed at high speed for from 5 to 30 minutes, under vacuum of from about 20 to 50 mm of Hg, preferably about 30 mm Hg. The resultant product is in each case a homogeneous, semi-solid, extrudable paste or gel product.

The following example further describes and demonstrates preferred embodiments within the scope of the present invention. The example is given solely for illustration, and are not to be construed as limitation of this invention as many variations thereof are possible without departing from its spirit and scope.

### EXAMPLE I

Toothpaste compositions containing ethyl lauroyl arginine HCl (ELAH) and a ethoxylated hydrogenated castor oil precoated coated silica abrasive and thickener were prepared having the following ingredients:

TABLE I

| | Composition (Wt. %) | | |
|---|---|---|---|
| Ingredients | A | B | C |
| Polyethylene glycol 600 | 3 | 3 | 3 |
| PEG-40 castor oil | 6 | 6 | 0 |
| Hydroxyethyl cellulose | 1.0 | 1.0 | 1.0 |
| Xanthan | 0.2 | 0.2 | 0.2 |
| Sodium saccharin | 0.35 | 0.35 | 0.35 |
| Sodium fluoride | 0.243 | 0.243 | 0.243 |
| Sorbitol | 40 | 40 | 40 |
| Sodium hydroxide, 50% soln. | 0.5 | 0.5 | 0.5 |
| Titanium dioxide | 0.5 | 0.5 | 0.5 |
| ELAH | 0.5 | 0 | 0.5 |
| Zeodent 115 | 5 | 5 | 5 |
| Zeodent 165 | 2 | 2 | 2 |
| Sylodent XWA 650 | 15 | 15 | 15 |
| Polysorbate 20 | 1 | 1 | 1 |
| Cocomidopropyl betaine | 1 | 1 | 1 |
| Flavor | 0.72 | 0.72 | 0.72 |
| Water to make | 100 | 100 | 100 |

The dentifrice “Composition A” was prepared by dispersing the sorbitol in the water in a conventional mixer under agitation. Into the dispersion was added the xanthan, PEG 40 castor oil, sodium fluoride, hydroxyethyl cellulose, and sodium saccharine. The resultant mixture was agitated until a homogeneous gel phase was formed. Into the gel phase was added TiO₂ and sodium hydroxide to adjust the pH to 6.5. These ingredients were mixed until a homogenous phase was obtained. The mixture was then transferred to a high speed/vacuum mixer; wherein the PEG 40 castor oil coated silica compounds Zeodent 115, Zeodent 165, and Sylodent XWA 650 were added and the mixture mixed at high speed for 25 minutes, under vacuum from about 30 mm Hg. Finally, polysorbate 20, cocoamidobetaine, flavor and ELAH were added to the mixture and mixed for an additional 10 minutes. The resultant product was a homogeneous, semisolid, extrudable paste or gel product.

For purposes of contrast, the procedure of the Example was repeated to prepare Composition B with the exception that ELAH was not included in the dentifrice formula. A second comparative composition, Composition C, was also prepared following the procedure of the Example with the exception that neither silica abrasive Zeodent 115 (Composition A) or the silica abrasive Zeodent 165 (Composition B) present in the dentifrice was coated with the PEG-40 castor oil.

The stability of the ELAH present in the prepared dentifrice composition A, B, C was measured by titrating a 0.015% wt. solution of the dentifrice with a 0.005N solution of sodium lauryl sulfate (SLS). The recovery results of ELAH as an indication of ELAH stability are recorded in Table II below.

TABLE II

| Composition | % Recovery ELAH |
|---|---|
| A | 87.1 |
| B | 3.0 |
| C | 7.5 |
| ELAH (Placebo) | 102.8 |

The antiplaque activity of Composition C was assessed using a flow cell model of the type disclosed in the Journal of Dental Research, vol. 73(II), pp. 1748-1755 (1994). Pooled human saliva was used as the bacterial source and single crystal germanium prisms as the oral surface model. Prior to exposure to bacteria, the surfaces were treated with a 2:1 dentifrice water slurry and then rinsed with artificial saliva (1 part porcine mucin 25 g/L, and 1 part saliva buffer solution) for 30 minutes under 1 mL/min flow conditions.

Composition A was assessed for overall plaque inhibition versus the comparative Composition B which did not contain ELAH, and Composition C in which the silica abrasive and thickener were not precoated with PEG 40 castor oil. The compositions were simultaneously run in the system. The lower the plaque score the more effective the antiplaque agent. The results recorded in Table III below show a significant reduction in plaque effected by Composition A when compared to comparative Compositions B and C.

TABLE III

| Composition | Plaque Index | % reduction |
|---|---|---|
| A | 1.4237 | 17.5 |
| B | 1.7265 | — |
| C | 1.6705 | 3.2 |

The results recorded in Table III indicate that Composition A containing the PEG-40 castor oil coated silica compounds was more effective in plaque reduction than Composition C which the silica compounds were not coated with the PEG 40 castor oil as well as Composition B which did not contain ELAH.

What is claimed is:

1. A stable antiplaque oral composition comprising an aqueous vehicle containing a safe and effective amount of an antibacterial arginine derivative compound represented by the 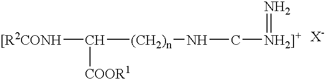
wherein R¹ is an alkyl chain of 1 to 8 carbon atoms, and R² is an alkyl chain of 6 to 30 carbon atoms and X is an anion,; and, a silica compound, the silica compound having been precoated with an ethoxylated hydrogenated castor oil:

2. The composition of claim 1 wherein n=3.

3. The composition according to claim 1 wherein the concentration of the arginine derivative compound is present in the oral composition at a concentration of about 0.02% to 2% by weight.

4. The composition according to claim 1 wherein the antibacterial arginine derivative compound is ethyl lauroyl arginine hydrochloride.

5. The composition of claim 1 wherein the silica compound is a silica abrasive.

6. The composition of claim 1 wherein the silica compound is a silica thickener.

7. The composition of claim 1 wherein the silica compound is precoated with an ethoxylated hydrogenated castor oil at a weight ratio of castor oil to silica of 1:10 to 1:2.
